(19) United States
(12) Patent Application Publication
(10) Pub. No.: US 2006/0281115 A1
(43) Pub. Date: Dec. 14, 2006

(54) MAX quadruplex nucleic acids and uses thereof

(75) Inventor: Adam Siddiqui-Jain, San Diego, CA (US)

Correspondence Address: MORRISON & FOERSTER LLP, 12531 HIGH BLUFF DRIVE, SUITE 100, SAN DIEGO, CA, 92130-2040, US

(73) Assignee: Cylene Pharmaceuticals, Inc., San Diego, CA (US)

(21) Appl. No.: 11/440,268

(22) Filed: May 24, 2006

Related U.S. Application Data
(60) Provisional application No. 60/684,498, filed on May 24, 2005.

(51) Int. Cl. C12Q 1/68; C07H 21/04

(52) U.S. Cl. 435/6; 536/24.3

(57) ABSTRACT

The MAX regulatory region contains a functional quadruplex structure. Thus, provided herein are MAX quadruplex nucleic acid acids, nucleic acid therapeutics that target quadruplex-altered nucleotide sequences and methods, methods for identifying compounds that modulate the biological activity of a native MAX quadruplex DNA, and methods for modulating the biological activity of a native MAX quadruplex DNA with a compound identified by the methods described herein.

### CROSS-REFERENCE TO RELATED APPLICATIONS

This application claims benefit of Provisional Application Ser. No. 60/684,498 filed May 24, 2005. The contents of this document are incorporated herein by reference.

### TECHNICAL FIELD

The invention relates to DNA capable for forming quadruplex secondary structure.

### BACKGROUND ART

Developments in molecular biology have led to an understanding of how certain therapeutic compounds interact with molecular components and lead to a modified physiological condition. Specificity of therapeutic compounds for their targets is derived in part from complementary structural elements between the target molecule and the therapeutic compound. A greater variety of target structural elements leads to the possibility of unique and specific target/compound interactions. In particular, researchers have identified compounds which target DNA. Some of these compounds are effective anticancer agents and have led to significant increases in the survival of cancer patients.

Unfortunately, however, these DNA targeting compounds do not specifically act on cancer cells and are therefore extremely toxic. One reason why DNA targeting compounds may be unspecific is DNA requires the uniformity of Watson-Crick duplex structure for compactly storing information within the human genome. This uniformity of DNA structure does not lead to a structurally diverse population of DNA molecules where each of the molecules can be specifically targeted.

Quadruplexes are secondary structure that can form in certain purine-rich strands of DNA molecules that have characteristic motifs within specific regulatory regions for DNA molecules. In duplex DNA molecules (or nucleic acids), certain purine rich strands are capable of engaging in a slow equilibrium between a typical duplex helix structure and in unwound and non-B-form regions. These unwound and non-B forms can be referred to as “paranemic structures.” Some forms are associated with sensitivity to S1 nuclease digestion, which can be referred to as “nuclease hypersensitivity elements” or “NHEs.” A quadruplex is one type of paranemic structure and certain NHEs can adopt a quadruplex structure. As a result, quadruplex forming regions of DNA offer a structural element that is specific for a particular regulatory region, and thus may be potential molecular targets for anticancer agents.

MAX is a basic-helix-loop-helix-leucine zipper (bHLHZip) protein capable of forming sequence-specific DNA binding complexes with members of the MYC family of proteins. As an obligate partner, MAX regulates MYC's ability to activate transcription, and promote cell proliferation, transformation, or apoptosis. MAX is constitutively and ubiquitously expressed and dimerizes with a number of other bHLHZip proteins including members of the Mad family including Mad1, Mxi1, Mad3, Mad4 as well as Mnt and Mga. These other binding partners appear to compete with MYC for MAX binding, highlighting the critical nature of the MYC-MAX interaction in cell growth and development.

### DISCLOSURE OF THE INVENTION

Certain regulatory regions in duplex DNA can transition into single stranded structures, including intrastrand quadruplex structures. Identifying quadruplex structures associated with genes involved with aberrant conditions (e.g., cell proliferative conditions) paves the way for identifying compounds that specifically interact with a quadruplex DNA structure in vivo and can treat diseases. Thus, a need exists for identifying biologically relevant quadruplex structures present in cellular DNA.

Accordingly, featured herein is a core quadruplex sequence in the MAX 5′ untranslated region located 3′ of the transcription start site. In particular, a substantially pure or isolated nucleic acid comprising a nucleotide sequence of SEQ ID NO:1, or portion thereof, is provided.

Also, featured herein is a method for identifying a compound that modulates the biological activity of a MAX quadruplex DNA comprising SEQ ID NO:1, which comprises contacting the quadruplex DNA with a candidate molecule, and determining the presence or absence of an interaction between the candidate molecule and the quadruplex DNA. One embodiment is a method for identifying a molecule that binds a MAX quadruplex DNA comprising SEQ ID NO:1, which comprises contacting the quadruplex DNA with a candidate molecule, and determining the presence or absence of binding between the candidate molecule and the quadruplex DNA. The candidate molecule often is a compound or a nucleic acid, such as an antisense, ribozyme, siRNA or RNAi nucleic acid, for example.

Also featured is a method for modulating the biological activity of a MAX quadruplex DNA comprising SEQ ID NO:1, which comprises contacting a system comprising said quadruplex DNA with a molecule which interacts with the quadruplex DNA.

The DNA of certain subjects may include an alteration in the MAX nucleotide sequence. The alteration can be an insertion, deletion, substitution or other modification in a nucleotide sequence 5′ of the MAX open reading frame that can form a quadruplex structure. Without being limited by theory, such an alteration may alter a quadruplex structure in MAX that regulates transcription or other cellular process. Thus, featured herein are prognostic methods for determining whether a subject is at risk of developing or having a cell proliferative disoder, such as cancer, by detecting an altered MAX quadruplex sequence in a DNA sample from the subject. In a related embodiment, featured herein is a method for identifying a subject at risk of developing or having cancer by detecting the presence or absence of an altered MAX quadruplex sequence in a DNA sample of the subject, and if an altered MAX quadruplex sequence is detected in the DNA sample from the subject, targeting cancer prevention and/or treatment regimens to the subject. In one embodiment, disclosed herein is an antisense nucleic acid cancer therapy that specifically targets DNA in subjects having an altered MAX quadruplex sequence.

Also featured herein is a method for selecting a subject for treatment of a disorder with a quadruplex-interacting molecule, which comprises: determining whether a nucleic acid from a subject comprises an altered MAX nucleotide sequence; and selecting a subject for treatment of a disorder based upon the presence or absence of the altered MAX nucleotide sequence. The disorder can be a cell proliferative disorder such as cancer or rheumatoid arthritis. Sometimes, the subject identified with a nucleic acid having an altered MAX nucleotide sequence is selected for treatment with the quadruplex-interacting molecule. In other embodiments, the subject identified with a nucleic acid not having an altered MAX nucleotide sequence is selected for treatment with the quadruplex-interacting molecule.

### MODES OF CARRYING OUT THE INVENTION

The present invention relates to the identification of a quadruplex DNA structures in the MAX promoter as a biologically relevant oncogene regulator. Thus, isolated MAX quadruplex-forming DNA is useful for screening molecules that interact with quadruplex structures to identify new treatments for cancer as well as other MAX-associated diseases. Provided herein are quadruplex-altered nucleic acids, methods for determining whether a subject is at risk of developing or having cancer, pharmacogenomic methods for targeting appropriate prevention or therapeutic regimens to subjects identifying as being at risk of developing or having cancer, methods for screening molecules that interact with native and altered quadruplex sequences, and therapeutic methods for treating cancers.

Quadruplex Nucleic Acids and Variants Thereof

The MAX quadruplex nucleic acid of the present invention may comprise or consist of a nucleotide sequence or a portion of a nucleotide sequence set forth below.

SEQ ID NO:1

5′-CGGCGGCGGGGAGGGGAAGGGGTGAAGGGGAGGGGGA-3

As used herein, the term “quadruplex nucleic acid” and “quadruplex forming nucleic acid” refers to a nucleic acid in which a quadruplex structure may form. Altered quadruplex sequences include those with mutations that alter the quadruplex in some way, sometimes destabilizing the quadruplex or even creating a new quadruplex. The entire length of the nucleic acid may participate in the quadruplex structure or a portion of the nucleic acid length may form a quadruplex structure. The term “test nucleic acid” as used herein refers to a nucleic acid that may or may not be capable of forming a quadruplex structure. A quadruplex nucleic acid may include one or more G-tetrad structures. In some embodiments, the quadruplex-forming nucleic acids described herein are capable of forming a parallel quadruplex structure having four parallel strands (e.g., propeller structure), antiparallel quadruplex structure having two stands that are antiparallel to the two parallel strands (e.g., chair or basket quadruplex structure) or a partially parallel quadruplex structure having one strand that is antiparallel to three parallel strands (e.g., a chair-eller or basket-eller quadruplex structure) (described in greater detail in U.S. Application Nos. 2004/0005601 and PCT Application PCT/US2004/037789.

Quadruplex nucleic acids and test nucleic acids may comprise or consist of DNA (e.g., genomic DNA (gDNA) and complementary DNA (cDNA)) or RNA (e.g., mRNA, tRNA, and rRNA). In embodiments where a quadruplex nucleic acid or test nucleic acid is a gDNA or cDNA fragment, the fragment is often 50 or fewer, 100 or fewer, or 200 or fewer base pairs in length, and is sometimes about 300, about 400, about 500, about 600, about 700, about 800, about 900, about 1000, about 1100, about 1200, about 1300, or about 1400 base pairs in length. Methods for generating gDNA and cDNA fragments are well known in the art (e.g., gDNA may be fragmented by shearing methods and cDNA fragment libraries are commercially available). In embodiments where the quadruplex nucleic acid or test nucleic acid is a synthetically prepared oligonucleotide, the oligonucleotides can be about 8 to about 80 nucleotides in length, often about 8 to about 50 nucleotides in length, and sometimes from about 10 to about 30 nucleotides in length. In other words, the oligonucleotide often is about 80 or fewer, about 70 or fewer, about 60 or fewer, or about 50 or fewer nucleotides in length, and sometimes is about 40 or fewer, about 35 or fewer, about 30 or fewer, about 25 or fewer, about 20 or fewer, or about 15 or fewer nucleotides in length. Synthetic oligonucleotides can be synthesized using standard methods and equipment, such as by using an ABI™3900 High Throughput DNA Synthesizer, which is available from Applied Biosystems (Foster City, Calif.).

Quadruplex nucleic acids and test nucleic acids may comprise or consist of analog or derivative nucleic acids, such as peptide nucleic acids (PNA) and others exemplified in U.S. Pat. Nos. 4,469,863; 5,536,821; 5,541,306; 5,637,683; 5,637,684; 5,700,922; 5,717,083; 5,719,262; 5,739,308; 5,773,601; 5,886,165; 5,929,226; 5,977,296; 6,140,482; WIPO publications WO 00/56746 and WO 01/14398, and related publications. Methods for synthesizing oligonucleotides comprising such analogs or derivatives are disclosed, for example, in the patent publications cited above, in U.S. Pat. Nos. 5,614,622; 5,739,314; 5,955,599; 5,962,674; 6,117,992; in WO 00/75372; and in related publications.

The MAX quadruplex nucleic acids or test nucleic acids utilized in the methods described herein sometimes include a nucleotide sequence that is substantially similar, but not identical, to the native genomic DNA nucleotide sequence disclosed herein. A core motif or subsequence may be present within the quadruplex sequence. Such a quadruplex nucleic acid or a test nucleic acid utilized in the present system can be in a quadruplex structural conformation described above.

Substantially similar quadruplex nucleic acids often are nearly identical to native quadruplex nucleotide sequences and sometimes include one or more altered MAX quadruplex sequences. Such alterations, which are also referred to hereafter as “polymorphisms,” may result from an insert, deletion, or substitution of one or more nucleotides. Substitutions can include a single nucleotide replacement of a guanine that participates in a G-tetrad, where one, two, three, or four of more of such guanines in the quadruplex nucleic acid are substituted. Methods for identifying quadruplex nucleotide sequences having altering guanine substitutions in different tissues and cells are described hereafter.

In one embodiment, the native MAX quadruplex nucleic acid can be converted to an altered nucleic acid by substituting one or more guanines that participate in a G-tetrad with another nucleotide (e.g. adenine). Such substitutions can be introduced by standard recombinant molecular biology techniques known in the art. One, two, three, or four or more guanines can be substituted in any quadruplex-forming nucleotide sequence, including the nucleotide sequences set forth herein, and as described in specific embodiments and examples hereafter. An altered nucleic acid often is about 80 or fewer, about 70 or fewer, about 60 or fewer, or about 50 or fewer nucleotides in length, and sometimes is about 40 or fewer, about 35 or fewer, about 30 or fewer, about 25 or fewer, about 20 or fewer, or about 15 or fewer nucleotides in length. Other alterations may be introduced, such as a nucleotide sequence insertion or deletion from the quadruplex nucleic acid of SEQ ID NO: 1.

Quadruplex nucleic acids and test nucleic acids may be contacted in the system as single-stranded nucleic acids, double stranded nucleic acids, or other forms of nucleic acids (see, e.g., Ren & Chaires, Biochemistry 38: 16067-16075 (1999)). Double stranded nucleic acids may be presented in the system by a plasmid, as exemplified herein.

Quadruplex nucleic acids can exist in different conformations, which differ in strand stoichiometry and/or strand orientation. See, e.g., U.S. Patent Application No. 2004/0005601. The ability of guanine rich nucleic acids of adopting these structural conformations is due to the formation of guanine tetrads through Hoogsteen hydrogen bonds. Thus, one nucleic acid sequence can give rise to different quadruplex orientations, where the different conformations depend in part upon the nucleotide sequence of the quadruplex nucleic acid and conditions under which they form, such as the concentration of potassium ions present in the system and the time that the quadruplex is allowed to form.

Different quadruplex conformations can be identified separately from one another using standard procedures known in the art, and as described herein. Also, multiple conformations can be in equilibrium with one another, and can be in equilibrium with duplex nucleic acid if a complementary strand exists in the system. The equilibrium may be shifted to favor one conformation over another such that the favored conformation is present in a higher concentration or fraction over the other conformation or other conformations. The term “favor” or “stabilize” as used herein refers to one conformation being at a higher concentration or fraction relative to other conformations. The term “hinder” or “destabilize” as used herein refers to one conformation being at a lower concentration. One conformation may be favored over another conformation if it is present in the system at a fraction of 50% or greater, 75% or greater, or 80% or greater or 90% or greater with respect to another conformation (e.g., another quadruplex conformation, another paranemic conformation, or a duplex conformation). Conversely, one conformation may be hindered if it is present in the system at a fraction of 50% or less, 25% or less, or 20% or less and 10% or less, with respect to another conformation.

Equilibrium may be shifted to favor one quadruplex form over another form by methods described herein. For example, certain bases in quadruplex DNA may be mutated to hinder or destabilize the formation of a particular conformation. Typically, these mutations are located in tetrad regions of the quadruplex (regions in which four bases interact with one another in a planar orientation). Also, ion concentrations and the time with which quadruplex DNA is contacted with certain ions can favor one conformation over another. For example, potassium ions stabilize quadruplex structures. The chair conformation is favored with contact times of 5 minutes or less in solutions containing 100 mM ions, and often contact times of 10 minutes or less, 20 minutes or less, 30 minutes or less, and 40 minutes or less. Ions, ion concentration and the counteranion can vary, and the skilled artisan can routinely determine which quadruplex conformation exists for a given set of conditions by utilizing the methods described herein. Furthermore, compounds that interact with quadruplex DNA may favor one form over the other and thereby stabilize one form.

Substantially Identical Nucleotide Sequences

Nucleotide sequences that are substantially identical to native quadruplex-forming nucleotide sequences are included herein. The term “substantially identical” refers to two or more nucleic acids sharing one or more identical nucleotide sequences. Included are nucleotide sequences that sometimes are 55%, 60%, 65%, 70%, 75%, 80%, or 85% identical to the native MAX quadruplex-forming nucleotide sequence, and often are 90% or 95% identical to the native quadruplex-forming nucleotide sequence (each identity percentage can include a 1%, 2%, 3% or 4% variance). One test for determining whether two nucleic acids are substantially identical is to determine the percentage of identical nucleotide sequences shared between the nucleic acids.

Calculations of sequence identity can be performed as follows. Sequences are aligned for optimal comparison purposes and gaps can be introduced in one or both of a first and a second nucleic acid sequence for optimal alignment. Also, non-homologous sequences can be disregarded for comparison purposes. The length of a reference sequence aligned for comparison purposes sometimes is 30% or more, 40% or more, 50% or more, often 60% or more, and more often 70%, 80%, 90%, 100% of the length of the reference sequence. The nucleotides at corresponding nucleotide positions then are compared among the two sequences. When a position in the first sequence is occupied by the same nucleotide as the corresponding position in the second sequence, the nucleotides are deemed to be identical at that position. The percent identity between the two sequences is a function of the number of identical positions shared by the sequences, taking into account the number of gaps, and the length of each gap, introduced for optimal alignment of the two sequences.

Comparison of sequences and determination of percent identity between two sequences can be accomplished using a mathematical algorithm. Percent identity between two nucleotide sequences can be determined using the algorithm of Meyers & Miller, CABIOS 4: 11-17 (1989), which has been incorporated into the ALIGN program (version 2.0), using a PAM120 weight residue table, a gap length penalty of 12 and a gap penalty of 4. Percent identity between two nucleotide sequences can be determined using the GAP program in the GCG software package (available at http address www.gcg.com), using a NWSgapdna.CMP matrix and a gap weight of 40, 50, 60, 70, or 80 and a length weight of 1, 2, 3, 4, 5, or 6. A set of parameters often used is a Blossum 62 scoring matrix with a gap open penalty of 12, a gap extend penalty of 4, and a frameshift gap penalty of 5.

Another manner for determining if two nucleic acids are substantially identical is to assess whether a polynucleotide homologous to one nucleic acid will hybridize to the other nucleic acid under stringent conditions. As use herein, the term “stringent conditions” refers to conditions for hybridization and washing. Stringent conditions are known to those skilled in the art and can be found in CURRENT PROTOCOLS IN MOLECULAR BIOLOGY, John Wiley & Sons, N.Y., 6.3.1-6.3.6 (1989). Aqueous and non-aqueous methods are described in that reference and either can be used. An example of stringent conditions is hybridization in 6× sodium chloride/sodium citrate (SSC) at about 45° C., followed by one or more washes in 0.2×SSC, 0. 1% SDS at 50° C. Another example of stringent conditions are hybridization in 6× sodium chloride/sodium citrate (SSC) at about 45° C., followed by one or more washes in 0.2×SSC, 0.1% SDS at 55° C. A further example of stringent conditions is hybridization in 6× sodium chloride/sodium citrate (SSC) at about 45° C., followed by one or more washes in 0.2×SSC, 0.1% SDS at 60° C. Often, stringent conditions are hybridization in 6× sodium chloride/sodium citrate (SSC) at about 45° C., followed by one or more washes in 0.2×SSC, 0.1% SDS at 65° C. Also, stringency conditions include hybridization in 0.5M sodium phosphate, 7% SDS at 65° C., followed by one or more washes at 0.2×SSC, 1% SDS at 65° C.

Identification of MAX Quadruplex Sequence Variants and Cancer Prognostics and Diagnostics

Specific quadruplex sequence alterations are associated with a risk of developing or having certain cancers and/or proliferation-related disorders. See Moshynska et al., J. Nat'l Inst. Cancer 96:673-82 (2004); U.S. Provisional Attorney Docket No. 53223-30021.00, filed Apr. 16, 2005. These data confirms the biological significance of quadruplex regulation as well as its potential role as a therapeutic target for anti-cancer agents. Moreover, MAX-MYC interactions server as critical regulators of cell growth and apoptosis in a variety of cells, and thus its dysregulation contributes to diseases and disorders such as cancer. Thus, the presence of an altering mutation in a MAX quadruplex sequence also may indicate an increased risk or presence of cancer.

Any type of mutation that alters a MAX quadruplex sequence may be associated with certain cancers and/or proliferation-related disorders. Typically, such mutations occur at polymorphic sites that alter quadruplex sequences. As used herein, the term “polymorphic site” refers to a region in a nucleic acid at which two or more alternative nucleotide sequences are observed, often in a significant number of nucleic acid samples from a population of individuals. These genetic alterations can occur at polymorphic sites that alter quadruplex structures, and may be are nucleotide substitutions from guanine to another nucleotide (e.g., adenine). A polymorphic site often is one nucleotide in length, which is referred to herein as a “single nucleotide polymorphism” or “SNP.” A polymorphic site also may be a nucleotide sequence of two or more nucleotides, an inserted nucleotide or nucleotide sequence, a deleted nucleotide or nucleotide sequence, or a microsatellite, for example.

Where there are two, three, or four alternative nucleotide sequences at a polymorphic site, each nucleotide sequence is referred to as a “mutant sequence,” “substituted sequence,” “polymorphic variant,” “nucleic acid variant,” or “allelic variant.” Where two polymorphic variants exist, for example, the polymorphic variant represented in a minority of samples from a population is sometimes referred to as a “minor allele” and the polymorphic variant that is more prevalently represented is sometimes referred to as a “major allele.” Many organisms possess two chromosomes where one is a near copy of the other (e.g., humans). Those individuals who possess the same allelic variants often are referred to as being “homozygous” and those individuals who possess different allelic variants normally are referred to as being “heterozygous.” Homozygous individuals sometimes are predisposed to a different phenotype as compared to heterozygous individuals. As used herein, the term “phenotype” refers to a trait which can be compared between individuals, such as presence or absence of a condition, a visually observable difference in appearance between individuals, a metabolic variation, a physiological variation, a variation in the function of a biological molecule, and the like. An example of a phenotype is occurrence of CLL or CML cancer.

The term “genotype” refers to a representation of an allelic variant in a subject of a population and the term “genotyped” refers to a method of detecting the presence or absence of a particular allelic variant in a subject of a population. A genotype or polymorphic variant may be expressed in terms of a “haplotype,” which as used herein refers to two or more polymorphic variants occurring on the same chromosome in a group of individuals within a population. For example, two SNPs may exist within a nucleotide sequence where each SNP position includes a cytosine variation and an adenine variation. Certain individuals in a population may carry one allele (heterozygous) or two alleles (homozygous) having a cytosine at each SNP position. As the two cytosines corresponding to each SNP in the gene travel together on one or both alleles in these individuals, the individuals can be characterized as having a cytosine/cytosine haplotype with respect to the two SNPs in the gene.

A polymorphic variant of the MAX regulatory sequence can be identified in any type of nucleic acid sample from any type of biological tissue or fluid. See, e.g., Akgul et al., Cell. Mol. Life Sci. 57:684-91 (2000). A nucleic acid sample typically is isolated from a biological sample obtained from a subject, and in specific embodiments, subjects diagnosed with cancer. For example, a nucleic acid sample can be isolated from blood, saliva, sputum, and urine, and often is isolated from a cell scraping or biopsy tissue sample (e.g. colorectal tissue) isolated from a subject having cancer. The nucleic acid sample can be isolated from a biological sample using standard techniques, such as described in Example 2. As used herein, the term “subject” primarily refers to humans but also sometimes refers to other mammals such as dogs, cats, and ungulates (e.g. cattle, sheep, and swine). Subjects also sometimes include avians (e.g. chickens and turkeys), reptiles, and fish (e.g. salmon), as methods described herein can be adapted to nucleic acid samples isolated from any of these organisms. The nucleic acid sample may be isolated from the subject and then directly utilized in a method for determining the presence of an allelic variant, or alternatively, the sample may be isolated and then stored (e.g. frozen) for a period of time before being subjected to analysis.

The presence or absence of an allelic variant is detected in one or both chromosomal complements represented in the nucleic acid sample. Determining the presence or absence of a polymorphic variant in both chromosomal complements represented in a nucleic acid sample is useful for determining the zygosity of the polymorphic variant (i.e. whether the subject is homozygous or heterozygous for the polymorphic variant). Any detection method known in the art may be utilized to determine whether a sample includes the presence or absence of a polymorphic variant described herein. While many detection methods include a process in which a DNA region carrying the polymorphic site of interest is amplified, ultrasensitive detection methods which do not require amplification may be utilized in the detection method, thereby eliminating the amplification process. Allelic variant detection methods known in the art include, for example, nucleotide sequencing methods (see e.g. Example 2); primer extension methods (U.S. Pat. Nos. 4,656,127; 4,851,331; 5,679,524; 5,834,189; 5,876,934; 5,908,755; 5,912,118; 5,976,802; 5,981,186; 6,004,744; 6,013,431; 6,017,702; 6,046,005; 6,087,095; 6,210,891; 5,547,835; 5,605,798; 5,691,141; 5,849,542; 5,869,242; 5,928,906; 6,043,031; 6,194,144; and 6,258,538; WO 01/20039; Chen & Kwok, Nucleic Acids Research 25: 347-353 (1997) and Chen et al, Proc. Natl. Acad. Sci. USA 94/20: 10756-10761 (1997)); ligase sequence determination methods (e.g., U.S. Pat. Nos. 5,679,524 and 5,952,174, and WO 01/27326); mismatch sequence determination methods (e.g., U.S. Pat. Nos. 5,851,770; 5,958,692; 6,110,684; and 6,183,958); microarray sequence determination methods; restriction fragment length polymorphism (RFLP) procedures; PCR-based assays (e.g., TAQMAN® PCR System (Applied Biosystems)); hybridization methods; conventional dot blot analyses; single strand conformational polymorphism analysis (SSCP, e.g., U.S. Pat. Nos. 5,891,625 and 6,013,499; Orita et al., Proc. Natl. Acad. Sci. U.S.A. 86: 27776-2770 (1989)); denaturing gradient gel electrophoresis (DGGE); heteroduplex analysis; mismatch cleavage detection; and techniques described in Sheffield et al., Proc. Natl. Acad. Sci. USA 49: 699-706 (1991), White et al., Genomics 12: 301-306 (1992), Grompe et al., Proc. Natl. Acad. Sci. USA 86: 5855-5892 (1989), and Grompe, Nature Genetics 5: 111-117 (1993). Those of skill in the art can utilize the determined nucleotide sequences flanking a polymorphic site in a database search to determine where the polymorphic site is located in genomic DNA.

A microarray can be utilized for determining whether a polymorphic MAX variant is present or absent in a nucleic acid sample. A microarray may include any oligonucleotide useful for detecting an altered MAX quadruplex sequence, and methods for making and using oligonucleotide microarrays suitable for use are disclosed in U.S. Pat. Nos. 5,492,806; 5,525,464; 5,589,330; 5,695,940; 5,849,483; 6,018,041; 6,045,996; 6,136,541; 6,142,681; 6,156,501; 6,197,506; 6,223,127; 6,225,625; 6,229,911; 6,239,273; WO 00/52625; WO 01/25485; and WO 01/29259. The microarray typically comprises a solid support and oligonucleotides may be linked to this solid support by covalent bonds or by non-covalent interactions. Oligonucleotides also may be linked to the solid support directly or by a spacer molecule.

In another embodiment, an integrated system is utilized for determining whether a polymorphic variant is present or absent in a nucleic acid sample. An example of an integrated system is a microfluidic system. These systems comprise a pattern of micro channels designed onto a glass, silicon, quartz, or plastic wafer included on a microchip. The movements of the samples are controlled by electric, electroosmotic or hydrostatic forces applied across different areas of the microchip. The microfluidic system may integrate nucleic acid amplification, sequencing, capillary electrophoresis and a detection method such as laser-induced fluorescence detection.

In yet another embodiment, a kit is utilized to identify a MAX genetic alteration in a sample. A kit often comprises one or more oligonucleotides useful for identifying an altered MAX quadruplex sequence, or a quadruplex motif or subsequence. Such oligonucleotides may amplify a fragment of genomic DNA having an altered MAX quadruplex sequence. The kit sometimes comprises a polymerizing agent, for example, a thermostable nucleic acid polymerase such as one disclosed in U.S. Pat. Nos. 4,889,818 or 6,077,664. Also, the kit often comprises chain elongating nucleotides, such as dATP, dTTP, dGTP, dCTP, and dITP, including analogs of dATP, dTTP, dGTP, dCTP and dITP, provided that such analogs are substrates for a thermostable nucleic acid polymerase and can be incorporated into a nucleic acid chain. The kit can include one or more chain terminating nucleotides such as ddATP, ddTTP, ddGTP, ddCTP, and the like. Kits optionally include buffers, vials, microtitre plates, and instructions for use.

In an embodiment, tissue samples are isolated from subjects diagnosed with a cancer and subjects diagnosed as not having the cancer, a nucleic acid sample is prepared from each tissue sample, and one or more quadruplex-forming nucleotide sequences are analyzed to identify an altered MAX quadruplex sequence associated with the cancer. The cancer can be any cancer, including but not limited to breast cancer; prostate cancer; lung cancer; lymphomas; skin cancer (e.g., basal cell carcinoma); pancreatic cancer; colorectal cancer; melanoma; ovarian cancer; non-small lung cancer; cervical carcinoma; leukemia (e.g., CLL, CML, anaplastic lymphoma kinase (ALK) positive anaplastic large cell lymphoma (ALCL); neuroblastoma; glioma; medulloblastoma, and astrocytoma. The isolated tissue is any located at or near the site affected by cancer, and sometimes is from a tumor or pre-malignant tissue (e.g., polyp), for example.

The tissue sample sometimes is frozen, placed in agar, cut into thin slices, and dissected (e.g., with a laser). The quadruplex-forming nucleic acid can have the sequence conforming to the sequences described above. Any of the methods for identifying a nucleotide substitutions described above can be utilized, and a standard nucleotide sequencing procedure preceded by a polymerase chain reaction procedure for amplifying the quadruplex-forming nucleotide sequence in the sample often is utilized, as described in Example 2 in connection with a cancer, for example. A nucleotide substitution is identified as associated with a cancer when it is present in a higher fraction of nucleic acid samples derived from subjects having cancer, and optionally, if the substitution is present in a significant fraction of the nucleic acid samples from subjects having cancer, for example, in nucleic acid samples from 5% or more, 10% or more, 15% or more, 20% or more, 25% or more, 30% or more, 40% or more, or 50% or more of the subjects having cancer.

Prognostic and diagnostic methods generally are directed to detecting the presence or absence of one or more genetic alterations in the MAX quadruplex sequence provided herein in a nucleic acid sample from a subject, where the presence of a particular genetic alteration determines that the subject is at risk of developing or having a cancer or other diseases disclosed herein. In specific embodiments, any of the foregoing detection methods may be utilized to prognose or diagnose a cancer associated with an altered MAX quadruplex sequence by detecting the presence of an altered MAX quadruplex sequence in a nucleic acid sample from a subject. Exemplary cancers include, but are not limited to adenocarcinoma, squamous carcinoma, leukemia, lymphoma, melanoma, sarcoma, or teratocarcinoma. Additionally, the cancer may be a cancer of the adrenal gland, bladder, bone, bone marrow, brain, breast, cervix, colon, gall bladder, ganglia, gastrointestinal tract, head and neck, heart, kidney, liver, lung, muscle, ovary, pancreas, parathyroid, penis, prostate, salivary glands, rectum, skin, spleen, testis, thymus, thyroid, or uterus.

In specific embodiments, the risk of a subject developing or having cancer can be determined by detecting the presence of a specific alteration in the MAX regulatory sequence. For a sequence complementary to the foregoing sequence, detecting a complementary alteration is probative of cancer risk. A subject may be heterozygous or homozygous with respect to the altered allele. A subject homozygous for the altered allele normally is at an increased risk of MAX-related cancer as compared to a subject homozygous for such an allele.

Predisposition to cancer or a related disorder can be expressed as a probability, such as an odds ratio, percentage, or risk factor. The predisposition is based upon the presence or absence of one or more altered MAX quadruplex sequences, and also may be based in part upon phenotypic traits of the individual being tested. Methods for calculating risk factors based upon patient data are well known. See e.g. Agresti, CATEGORICAL DATA ANALYSIS, 2nd Ed. (Wiley 2002).

Results from prognostic and diagnostic tests may be combined with other test results to diagnose, prevent, and treat cancer, as described in greater detail hereafter. Cancer prognostic and diagnostic methods tests sometimes are applied to nucleic acid samples derived from different subjects having varying stages of a particular cancer or other disease and sometimes are applied to nucleic acid samples derived from tissue samples representative of varying stages of a particular sample. In cancer, for example, the presence of an altered MAX quadruplex sequence is detected in nucleic acid samples corresponding to different stages of cancer and the presence of the allelic variant then is associated with one or more stages of the cancer for a diagnostic test.

In specific embodiments, results from diagnostic tests may be used to identify cancers that will be resistant to certain forms of chemotherapeutic and/or radiation therapy because of enhanced resistance to apoptotic stimuli.

Applications of Prognostic and Diagnostic Test Results

Pharmacogenomics is a discipline that involves tailoring a treatment for a subject according to the subject's genotype, as a particular treatment regimen may exert a differential effect depending upon the subject's genotype. Based upon the outcome of a prognostic or diagnostic test described herein, a clinician or physician may target a preventative or therapeutic treatment to a subject who would be benefited and avoid directing such a treatment to a subject who would not be benefited (e.g., the treatment has no therapeutic effect and/or the subject experiences adverse side effects).

The prognostic and diagnostic methods described herein are applicable to methods for preventing and treating cancer. For example, a nucleic acid sample from an individual may be subjected to a prognostic/diagnostic test described herein. Where one or more altered MAX quadruplex sequences associated with increased risk of cancer are identified in that subject, other diagnostic methods then may be ordered to characterize the progression of the cancer, and/or one or more cancer preventative regimens or treatment regimens then may be prescribed to that subject. The cancer preventative regimen or treatment regimen may be a general anticancer therapeutic (e.g. chemotherapeutic) or be allele-specific (e.g. antisense therapeutic).

For example, a subject identified by the prognostic or diagnostic procedures described above as having one or more alterations in the MAX quadruplex sequence can be identified as being at risk of developing or having cancer, and the necessary diagnostic procedure then may be ordered. In the event the scoping procedure identifies only pre-malignant or in situ maligancy, the tissue may be removed surgically or otherwise treated, thereby decreasing the probability that a more advanced stage of cancer manifests. Also, a biopsy or tissue scraping procedure may be prescribed and the tissue sample can be analyzed for the presence of cancerous cells. Thus, such a method allows for early detection and prevention of cancer.

In the event that an altered MAX quadruplex sequence is detected, a diagnostic procedure is ordered and completed, and tumors are detected by the scope procedure, a therapeutic treatment regimen for removing, shrinking or minimizing tumor growth can be prescribed to the subject. Such therapeutic treatment regimens include surgical removal of a tumor or tumors, biotherapy, chemotherapy, and/or radiation treatment, and these therapies can be carried out in any order or combination. For example, surgical removal often is followed by chemotherapy (e.g. fluorouracil, irinotecan, oxaliplatin), and sometimes chemotherapy is used in combination with radiation therapy to decrease colorectal tumor size before surgical removal of the tumor. These strategies are employed for earlier treatment of cancer, thereby enhancing the possibility of recovery.

In addition to the general therapeutic treatment regimens described above, allele-specific treatment regimens also may be proscribed to subjects determined to require the therapeutic based upon prognostic or diagnostic test results. In certain embodiments, a prognostic or diagnostic test described herein is used to detect a altered MAX quadruplex sequence associated with cancer in the DNA of a subject, and for subjects having a cancer associated allele, a molecule that interacts and often specifically interacts with the altered MAX nucleic acid is administered to the subject. In an embodiment, a peptide nucleic acid (PNA) molecule that specifically hybridizes to the MAX allele is administered to the subject to treat the cancer, as described in greater detail hereafter.

In specific embodiments, cancers identified as having a mutation in a MAX quadruplex sequence can be identified as those that are likely to benefit from combination therapeutic approaches. For example, in such cancers, compounds that modulate the MAX quadruplex can be combined with other agents that induce apoptosis such as avastin, dacarbazine (e.g., multiple myeloma), 5-FU (e.g., pancreatic cancer), gemcitabine (e.g., pancreatic cancer), and gleevac (e.g., CML).

In a specific embodiment, a compound of formulas I and II shown below can be administered to a system that includes a MAX quadruplex nucleotide sequence. Such a compound may interact with a nucleic acid comprising a MAX quadruplex nucleotide sequence, and may arrest a polymerase, such as RNA pol II. Such a compound also may be administered to subjects, including those diagnosed as having or not having an altered MAX quadruplex sequence.

In one aspect, the compounds have formula I, or pharmaceutically acceptable salts thereof

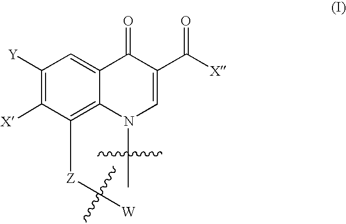

where X′ is hydroxy, alkoxy, carboxyl, halogen, CF₃, amino, amido, sulfide, 3-7 membered carbocycle or heterocycle, 5- or 6-membered aryl or heteroaryl, fused carbocycle or heterocycle, bicyclic compound, NR¹R², NCOR³, N(CH₂)ₙNR¹R², or N(CH₂)ₙR³, where the N in N(CH₂)ₙNR¹R² and N(CH₂)ₙR³ is optionally linked to a C1-10 alkyl, and each X′ is optionally linked to one or more substituents;

X″ is hydroxy, alkoxy, amino, amido, sulfide, 3-7 membered carbocycle or heterocycle, 5- or 6-membered aryl or heteroaryl, fused carbocycle or heterocycle, bicyclic compound, NR¹R², NCOR³, N(CH₂)ₙNR¹R², or N(CH₂)ₙR³, where the N in N(CH₂)ₙNR¹R² and N(CH₂)ₙR³ is optionally linked to a C1-10 alkyl, and X″ is optionally linked to one or more substituents;
Y is H, amino, halogen, or CF₃;
R¹, R² and R³ are independently H, C1-C6 alkyl, C1-C6 substituted alkyl, C3-C6 cycloalkyl, C1-C6 alkoxyl, carboxyl, imine, guanidine, 3-7 membered carbocycle or heterocycle, 5- or 6-membered aryl or heteroaryl, fused carbocycle or heterocycle, or bicyclic compound, where each R¹, R² and R³ are optionally linked to one or more substituents;

Z is CH₂, O, S, or NH;

and W is alkenyl, substituted alkenyl,

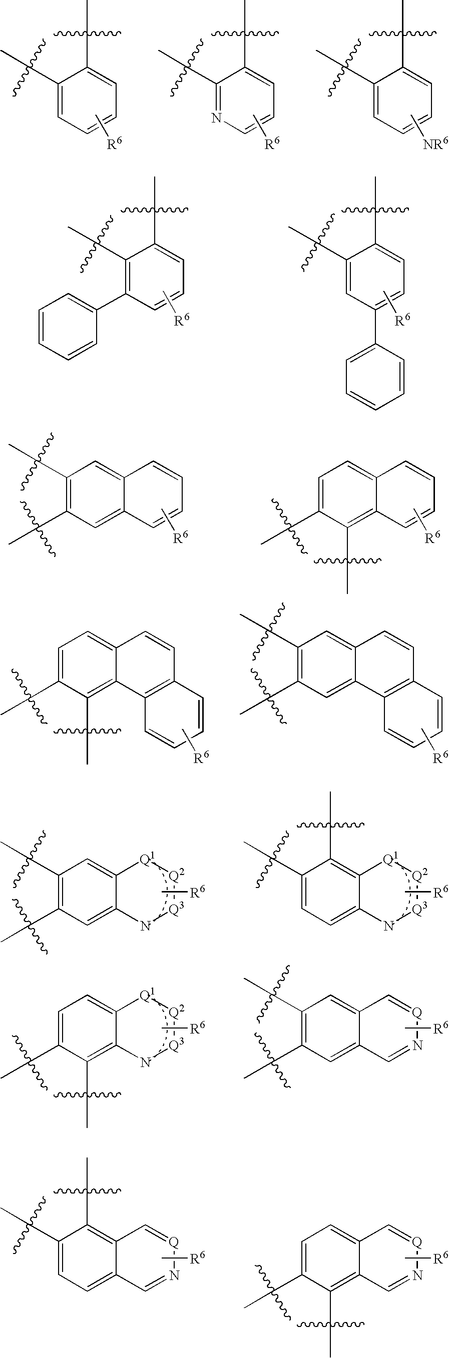

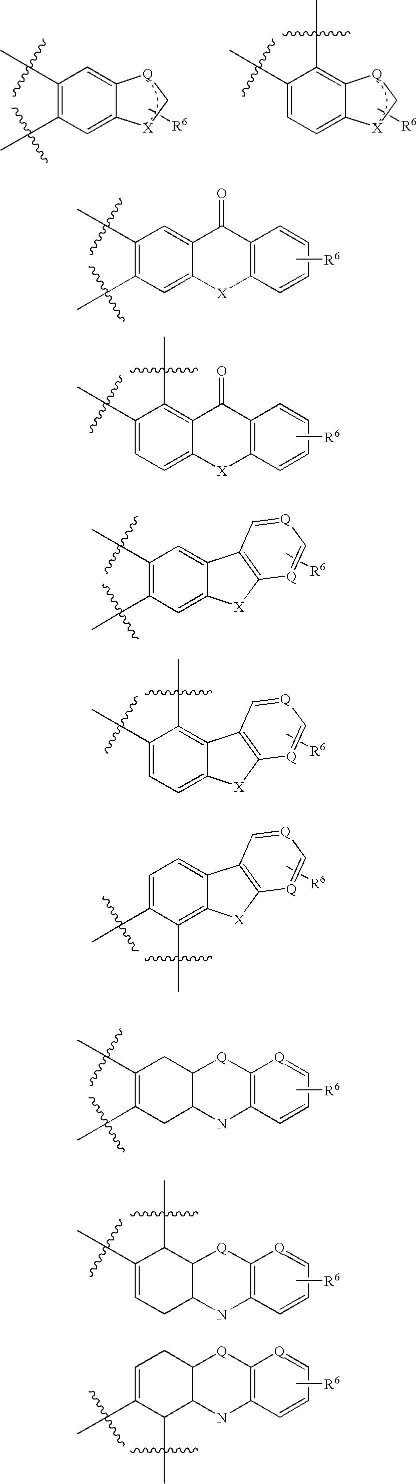

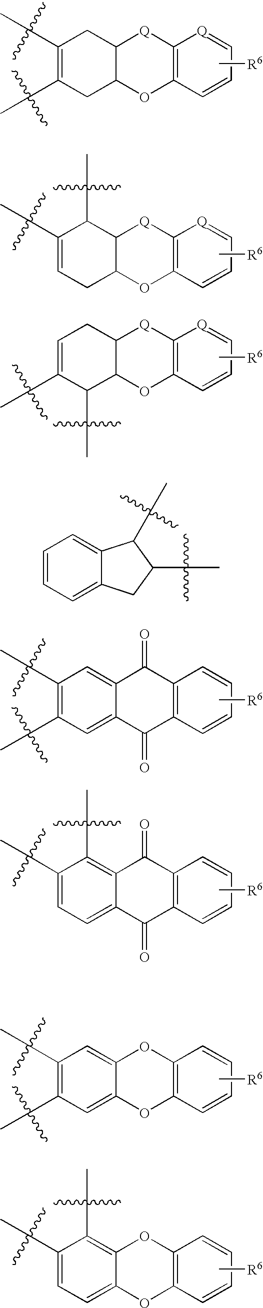

where R⁶ is H, hydroxyl, halogen, cyano, nitro, SH, C1-C10 alkyl, C1-C10 alkoxy, C1-C10 alkenyl, C2-C10 alkynyl, C3-C8 cycloalkyl where one or more carbons may be replaced with O or N, C5-C10 cycloalkenyl, NR¹R², COR⁷, OCOR⁷, CONR⁷(CH₂)nR¹R², NR⁷COR⁷, N(COR⁷)₂, NR⁷CONHR⁷, OR⁷, SOR⁷;

R⁷ is H, C1-C6 alkyl, C3-C6 cycloalkyl, or aryl;

n=0-6;

each of Q, Q¹, Q² and Q³ is independently CH, O or N;

X is (CH₂)ₘ, CO, O or N;

m=0-1;

and with the provisos that X′ is not 3-aminopyrrolidine, 3-amidopyrrolidine or 2-aminopyrrolidine when Y is F, Z is O, W is napthalenyl, phenyl, or methoxyphenyl, and X″ is hydroxyl or alkoxy;

X′ is not 3-aminopyrrolidine when Y is F, Z is O, W is benzyl, and X″ is 2,4-difluoroaniline or morpholinyl;

X′ is not piperazine when X″ is hydroxy, Y is F, Z is S, and W is phenyl;

X′ is not piperazine or methyl piperazine when X″ is hydroxy, Y is F, Z is O, and W is aniline or nitrobenzene;

X″ is not morpholinyl or 2,4-difluoroaniline when X′ and Y are F, Z is O, and W is benzyl; and

X″ is not hydroxyl or alkoxy when Y is H or halogen, Z is CH₂, O, S, W is phenyl or substituted phenyl, and X′ is halogen, pyridyl, pyrrolidine, piperidine, diazepine or amino.

In the above formula I, W may be benzene, pyridine, biphenyl, napthalene, phenanthrene, quinoline, isoquinoline, quinazoline, cinnoline, phthalazine, quinoxaline, indole, benzimidazole, benzoxazole, benzthiazol, benzofuran, anthrone, xanthone, acridone, fluorenone, carbazole, pyrimido[4,3-b]furan, pyrido[4,3-b]indole, pyrido[2,3-b]indole, dibenzofuran, acridine, and acridizine.

In one embodiment, the compound is has a formula (1A), 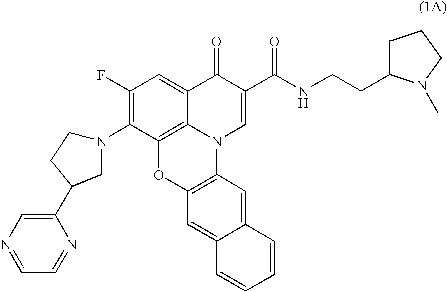
and pharmaceutically acceptable salts, esters and prodrugs thereof.

In another aspect, the compounds have formula II, or pharmaceutically acceptable salts thereof

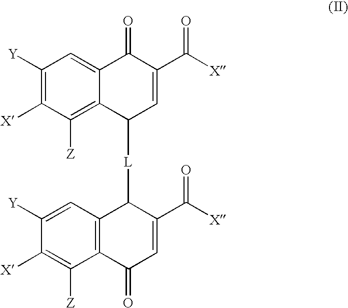

where X′ is hydroxy, alkoxy, carboxyl, halogen, CF₃, amino, amido, sulfide, 3-7 membered carbocycle or heterocycle, 5- or 6-membered aryl or heteroaryl, fused carbocycle or heterocycle, bicyclic compound, NR¹R², NCOR³, N(CH₂)ₙNR¹R², or N(CH₂)ₙR³, where the N in N(CH₂)ₙNR¹R² and N(CH₂)ₙR³ is optionally linked to a C1-10 alkyl, and each X′ is optionally linked to one or more substituents;

X″ is hydroxy, alkoxy, amino, amido, sulfide, 3-7 membered carbocycle or heterocycle, 5- or 6-membered aryl or heteroaryl, fused carbocycle or heterocycle, bicyclic compound, NR¹R², NCOR³, N(CH₂)ₙNR¹R², or N(CH₂)ₙR³, where the N in N(CH₂)ₙNR¹R² and N(CH₂)ₙR³ is optionally linked to a C1-10 alkyl, and X″ is optionally linked to one or more substituents;

Y is H, halogen, or CF₃;

R¹, R² and R³ are independently H, C1-C6 alkyl, C1-C6 substituted alkyl, C3-C6 cycloalkyl, C1-C6 alkoxyl, carboxyl, imine, guanidine, 3-7 membered carbocycle or heterocycle, 5- or 6-membered aryl or heteroaryl, fused carbocycle or heterocycle, or bicyclic compound, where each R¹, R² and R³ are optionally linked to one or more substituents;

Z is a halogen;

and L is a linker having the formula Ar¹—L1—Ar², where Ar1 and Ar2 are aryl or heteroaryl.

In the above formula II, L1 may be (CH₂)ₘ where m is 1-6, or a heteroatom optionally linked to another heteroatom such as a disulfide. Each of Ar1 and Ar2 may independently be aryl or heteroaryl, optionally substituted with one or more substituents. In one example, L is a [phenyl-S-S-phenyl] linker linking two quinolinone. In a particular embodiment, L is a [phenyl-S-S-phenyl] linker linking two identical quinoline species (see e.g., compound 121 in FIG. 1).

In the above formula I and II, X″ is hydroxy, alkoxy, amino, amido, sulfide, 3-7 membered carbocycle or heterocycle, 5- or 6-membered aryl or heteroaryl, fused carbocycle or heterocycle, bicyclic compound, NR¹R², NCOR³, N(CH₂)ₙNR¹R², or N(CH₂)ₙR³, where the N in N(CH₂)ₙNR¹R² and N(CH₂)ₙR³ is optionally linked to a C1-10 alkyl, and X″ is optionally linked to one or more substituents.

The invention further contemplates the use of the compounds disclosed in U.S. Application Nos. 2004/0005601, PCT Application PCT/US2004/037789, U.S. application Ser. Nos. 10/820,487, filed Apr. 7, 2004; 10/821,243, filed Apr. 7, 2004; 10/903,975, filed Jul. 20, 2004; 60/611,030, filed Sep. 17, 2004; 60/638,603, filed Dec. 22, 2004; 60/671,760, filed Apr. 14, 2005; and 60/671,617, filed Apr. 16, 2005; and incorporates the disclosure of each of these application in their entirety.

Quadruplex-Interacting Molecules

Native MAX quadruplex nucleic acids and variants thereof (e.g. a nucleic acid having an altered MAX quadruplex sequence) are utilized to screen for molecules that specifically interact with quadruplex structures. In these screening assays, one or more candidate molecules (also referred to as “test molecules” or “test compounds”) may be added to a system, where test molecules and quadruplex nucleic acids can be added to the system in any order. For example, a test molecule may be added to a system after a MAX nucleic acid is added; a test molecule may be added to a system before a MAX nucleic acid is added; or a test molecule may be added simultaneously to a system with a nucleic acid. A MAX quadruplex nucleic acid often is added to a system and then a test molecule is added.

Quadruplex interacting molecules typically interact with quadruplexes by reversible binding, and can stabilize already formed quadruplex structures or act as a template for generating quadruplex structures. Quadruplex interacting molecules often exhibit a hyperbolic relationship when biological activity is plotted as a function of quadruplex interacting molecule concentration. The quadruplex interacting molecule sometimes increases or decreases the biological activity being monitored. In addition to reversible binding, test molecules may interact with nucleic acids with irreversible binding, by cleaving one or more strands of a nucleic acid, or by adding chemical moieties to the nucleic acid (e.g., alkylation), for example, depending upon the structure and function of the test molecule.

Test molecules sometimes are organic or inorganic compounds having a molecular weight of 10,000 grams per mole or less, and sometimes having a molecular weight of 5,000 grams per mole or less, 1,000 grams per mole or less, or 500 grams per mole or less. Also included are salts, esters, and other pharmaceutically acceptable forms of the compounds. Compounds that interact with nucleic acids are known in the art. See, e.g. Hurley, Nature Rev. Cancer 2, 188-200 (2002); Anantha et al., Biochemistry Vol. 37, No. 9: 2709-2714 (1998); and Ren et al., Biochemistry 38: 16067-16075 (1999).

Compounds can be obtained using any of the combinatorial library methods known in the art, including spatially addressable parallel solid phase or solution phase libraries; synthetic library methods requiring deconvolution; “one-bead one-compound” library methods; and synthetic library methods using affinity chromatography selection. Examples of methods for synthesizing molecular libraries are described, for example, in DeWitt et al., Proc. Natl. Acad. Sci. U.S.A. 90: 6909 (1993); Erb et al., Proc. Natl. Acad. Sci. USA 91: 11422 (1994); Zuckermann et al., J. Med. Chem. 37: 2678 (1994); Cho et al., Science 261: 1303 (1993); Carrell et al., Angew. Chem. Int. Ed. Engl. 33: 2059 (1994); Carell et al., Angew. Chem. Int. Ed. Engl. 33: 2061 (1994); and Gallop et al., J. Med. Chem. 37: 1233 (1994).

In addition to an organic and inorganic compound, a test molecule is sometimes a nucleic acid, an antisense nucleic acid (described in more detail hereafter), a catalytic nucleic acid (e.g., a ribozyme), a nucleotide, a nucleotide analog, a polypeptide, an antibody, or a peptide mimetic. Methods for making and using these test molecules are known in the art. For example, methods for making ribozymes and assessing ribozyme activity are described (see, e.g., U.S. Pat. Nos. 5,093,246; 4,987,071; and 5,116,742; Haselhoff & Gerlach, Nature 334: 585-591 (1988) and Bartel & Szostak, Science 261: 1411-1418 (1993)). Also, peptide mimetic libraries are described (see, e.g., Zuckermann et al., J. Med. Chem. 37: 2678-85 (1994)).

Systems and Solid Supports

In assays that detect the presence or absence of an interaction between a quadruplex nucleic acid and a quadruplex-interacting molecule, test molecules are contacted with a nucleic acid in a system. As used herein, the term “contacting” refers to placing a signal molecule and/or a test molecule in close proximity to a quadruplex nucleic acid or test nucleic acid and allowing the molecules to collide with one another by diffusion. Contacting these assay components with one another can be accomplished by adding assay components to one body of fluid or in one reaction vessel, for example. The components in the system may be mixed in variety of manners, such as by oscillating a vessel, subjecting a vessel to a vortex generating apparatus, repeated mixing with a pipette or pipettes, or by passing fluid containing one assay component over a surface having another assay component immobilized thereon, for example.

As used herein, the term “system” refers to an environment that receives the assay components, which includes, for example, microtitre plates (e.g., 96-well or 384-well plates), silicon chips having molecules immobilized thereon and optionally oriented in an array (e.g., described above and in U.S. Pat. No. 6,261,776 and Fodor, Nature 364: 555-556 (1993)), and microfluidic devices (e.g., described above and in U.S. Pat. Nos. 6,440,722; 6,429,025; 6,379,974; and 6,316,781). The system can include attendant equipment for carrying out the assays, such as signal detectors, robotic platforms, and pipette dispensers.

One or more assay components may be immobilized to a solid support. The attachment between an assay component and the solid support may be covalent or non-covalent (see, e.g., U.S. Pat. No. 6,022,688 for non-covalent attachments). The solid support may be one or more surfaces of the system, such as one or more surfaces in each well of a microtiter plate, a surface of a silicon wafer, a surface of a bead (see e.g. Lam, Nature 354: 82-84 (1991)) that is optionally linked to another solid support, or a channel in a microfluidic device, for example. Types of solid supports, linker molecules for covalent and non-covalent attachments to solid supports, and methods for immobilizing nucleic acids and other molecules to solid supports are known (see e.g. U.S. Pat. Nos. 6,261,776; 5,900,481; 6,133,436; and 6,022,688; and WIPO publication WO 01/18234).

In an embodiment, polypeptide test molecules may be linked to a phage via a phage coat protein. The latter embodiment is often accomplished by using a phage display system, where quadruplex nucleic acids linked to a solid support are contacted with phages that display different polypeptide test molecules. Phages displaying polypeptide test molecules that interact with the immobilized nucleic acids adhere to the solid support, and phage nucleic acids corresponding to the adhered phages are then isolated and sequenced to determine the sequence of the polypeptide test molecules that interacted with the immobilized nucleic acids. Methods for displaying a wide variety of peptides or proteins as fusions with bacteriophage coat proteins are well known (Scott and Smith, Science 249: 386-390 (1990); Devlin, Science 249: 404-406 (1990); Cwirla et al., Proc. Natl. Acad. Sci. 87: 6378-6382 (1990); Felici, J. Mol. Biol. 222: 301-310 (1991)). Methods are also available for linking the test polypeptide to the N-terminus or the C-terminus of the phage coat protein. The original phage display system was disclosed, for example, in U.S. Pat. Nos. 5,096,815 and 5,198,346. This system used the filamentous phage M13, which required that the cloned protein be generated in E. coli and required translocation of the cloned protein across the E. coli inner membrane. Lytic bacteriophage vectors, such as lambda, T4 and T7 are more practical since they are independent of E. coli secretion. T7 is commercially available and described in U.S. Pat. Nos. 5,223,409; 5,403,484; 5,571,698; and 5,766,905.

Identifying MAX Quadruplex Interacting Molecules

Test molecules often are identified as quadruplex interacting molecules where a biological activity of the quadruplex, often expressed as a “signal,” produced in a system containing the test molecule is different than the signal produced in a system not containing the test molecule. Also, test nucleic acids are identified as quadruplex forming nucleic acids when the signal detected in a system that includes the test nucleic acid is different than the signal detected in a system that does not include the test nucleic acid. While background signals may be assessed each time a new molecule is probed by the assay, detecting the background signal is not required each time a new molecule is assayed.

In addition to determining whether a test molecule or test nucleic acid gives rise to a different signal, the affinity of the interaction between the nucleic acid and test molecule or signal molecule may be quantified. IC₅₀, K_{d}, or Kᵢ threshold values may be compared to the measured IC₅₀ or K_{d} values for each interaction, and thereby identify a test molecule as a quadruplex interacting molecule or a test nucleic acid as a quadruplex forming nucleic acid. For example, IC₅₀ or K_{d} threshold values of 10 μM or less, 1 μM or less, and 100 nM or less are often utilized, and sometimes threshold values of 10 nM or less, 1 nM or less, 100 pM or less, and 10 pM or less are utilized to identify quadruplex interacting molecules and quadruplex forming nucleic acids.

Many assays are available for identifying quadruplex interacting molecules and quadruplex forming nucleic acids. In some of these assays, the biological activity is the quadruplex nucleic acid binding to a molecule and binding is measured as a signal. In other assays, the biological activity is a polymerase arresting function of a quadruplex and the degree of arrest is measured as a decrease in a signal. In certain assays, the biological activity is transcription and transcription levels can be quantified as a signal. In another assay, the biological activity is cell death and the number of cells undergoing cell death is quantified. See, e.g., Studzinski (ed.), APOPTOSIS: A PRACTICAL APPROACH (Oxford University Press 1999). Another assay monitors proliferation rates of cancer cells. Examples of assays are fluorescence binding assays, gel mobility shift assays (see, e.g., Jin & Pike, Mol. Endocrinol. 10: 196-205 (1996)), polymerase arrest assays, transcription reporter assays, cancer cell proliferation assays, and apoptosis assays (see, e.g., Amersham Biosciences (Piscataway, N.J.)), and embodiments of such assays are described hereafter and in Example 1. Also, topoisomerase assays can be utilized to determine whether the quadruplex interacting molecules have a topoisomerase pathway activity (see e.g. TopoGEN, Inc. (Columbus, Ohio)).

An example of a fluorescence binding assay is a system that includes a quadruplex nucleic acid, a signal molecule, and a test molecule. The signal molecule generates a fluorescent signal when bound to the quadruplex nucleic acid (e.g. N-methylmesoporphyrin IX (NMM)), and the signal is altered when a test molecule competes with the signal molecule for binding to the quadruplex nucleic acid. An alteration in the signal when test molecule is present as compared to when test molecule is not present identifies the test molecule as a quadruplex interacting molecule.

An example of an arrest assay is a system that includes a template nucleic acid, which may comprise a quadruplex forming sequence, and a primer nucleic acid which hybridizes to the template nucleic acid 5′ of the quadruplex-forming sequence. The primer is extended by a polymerase (e.g., Taq polymerase), which advances from the primer along the template nucleic acid. In this assay, a quadruplex structure can block or arrest the advance of the enzyme, leading to shorter transcription fragments. Also, the arrest assay may be conducted at a variety of temperatures, including 45° C. and 60° C., and at a variety of ion concentrations.

An a transcription reporter assay, test quadruplex DNA may be coupled to a reporter system, such that a formation or stabilization of a quadruplex structure can modulate a reporter signal. An example of such a system is a reporter expression system in which a polypeptide, such as luciferase or green fluorescent protein (GFP), is expressed by a gene operably linked to the potential quadruplex forming nucleic acid and expression of the polypeptide can be detected. As used herein, the term “operably linked” refers to a nucleotide sequence which is regulated by a sequence comprising the potential quadruplex forming nucleic acid. A sequence may be operably linked when it is on the same nucleic acid as the quadruplex DNA, or on a different nucleic acid. An exemplary luciferase reporter system is described herein.

In a cancer cell proliferation assay, cell proliferation rates are assessed as a function of different concentrations of test quadruplex interacting molecules added to the cell culture medium. Any cancer cell type can be utilized in the assay. In one embodiment, CLL cancer cells are cultured in vitro and test quadruplex-interacting molecules are added to the culture medium at varying concentrations.

Nucleic Acid Therapeutics Targeted to an MAX Allele

Provided herein are nucleic acid therapeutics that specifically interact with a MAX allele, such as an allele without an insert or an allele with an insert, or both, for example. The therapeutic may be any nucleic acid therapeutic, such as an antisense nucleic acid, a ribozyme, an siRNA or an RNAi, for example. In one embodiment, antisense nucleic acids are designed to hybridize to an allele having an altered MAX quadruplex sequence. The nucleotide sequence of the antisense nucleic acid is designed around one or more altered MAX quadruplex sequences. For example, upon identification of an altered MAX quadruplex sequence in any of the native quadruplex nucleic acids described above, a nucleic acid complementary to the altered nucleic acid is generated that includes a sequence complementary to the altered site. The therapeutic nucleic acid may be any length that allows hybridization to the target nucleotide sequence in vivo. The nucleic acid therapeutics sometimes are about 7, about 8, about 9, or about 10 nucleotides in length, often are about 12 or fewer, about 15 or fewer, about 17 or fewer, or about 20 or fewer nucleotides in length, and sometimes are about 25 or fewer, about 30 or fewer, about 40 or fewer, or about 50 or fewer nucleotides in length.

The quadruplex-targeted nucleic acid often is synthesized having a backbone with fewer negative charges as compared to a DNA backbone. Examples of such nucleic acids are peptide nucleic acids (PNA) and PNA molecules having amino acid side chain moieties (e.g. lysine, arginine, and histidine side chain moieties. See e.g. U.S. patent application publication no. 20020188101 (Neilsen et al.). In an embodiment, the nucleic acid is a PNA optionally linked at the C-terminus to a lysine moiety. In another embodiment, the PNA is conjugated to another peptide that facilitates transduction of the conjugate into cells. Examples of such transduction peptides are HIV tat peptides (see e.g. SEQ ID NOs: 2-7 of U.S. Pat. No. 5,652,122) and Antennapedia homeodomain peptides (e.g. GGRQIWFQNRMKWKK, GGLWFQNRMKWKKEN, GGGRQIKIWFQNRRMKWKK, or GGGKIWFQNRRMKWKKEN reported in Simmons et al., Bioorg. Med. Chem. Lttrs. 7: 3001-3006 (1997)). The transduction peptide often is linked to the N-terminal or C-terminal end of the DNA using standard techniques. When the peptide is attached to the C-terminus of the PNA, the PNA often will not include a C-terminal lysine moiety.

The quadruplex-targeted nucleic acid often is tested in vitro to determine the degree to which it hybridizes to a nucleic acid corresponding to a native quadruplex nucleotide sequence or allele with an altered MAX quadruplex sequence. A fluorescence binding assay or circular dichroism assay (described hereafter) can be utilized to determine whether the quadruplex-targeted nucleic acid hybridizes to the target nucleic acid. Upon a determination that the quadruplex targeting nucleic acid is functional in vitro, the nucleic acid often is screened in vivo in animal models or in human subjects and the effect on cancer is monitored.

The quadruplex-targeted nucleic acid can be administered in vitro or in vivo as a composition of a pharmaceutically acceptable salt, ester, or salt of such ester. The quadruplex-targeted nucleic acid can be formulated as naked polynucleotide (e.g. polynucleotide formulated in phosphate buffered saline) or it can be formulated with other components.

Compositions comprising a quadruplex-targeted nucleic acid can be prepared as a solution, emulsion, or polymatrix-containing formulation (e.g., liposome and microsphere). Examples of such compositions are set forth in U.S. Pat. Nos. 6,455,308 (Freier), 6,455,307 (McKay et al.), 6,451,602 (Popoff et al.), and 6,451,538 (Cowsert), and examples of liposomes also are described in U.S. Pat. No. 5,703,055 (Feigner et al.) and Gregoriadis, Liposome Technology vols. I to III (2nd ed. 1993). The compositions can be prepared for any mode of administration, including topical, oral, pulmonary, parenteral, intrathecal, and intranutrical administration. Examples of compositions for particular modes of administration are set forth in U.S. Pat. Nos. 6,455,308 (Freier), 6,455,307 (McKay et al.), 6,451,602 (Popoff et al.), and 6,451,538 (Cowsert). Quadruplex-targeted nucleic acid compositions may include one or more pharmaceutically acceptable carriers, excipients, penetration enhancers, and/or adjuncts. Choosing the combination of pharmaceutically acceptable salts, carriers, excipients, penetration enhancers, and/or adjuncts in the composition depends in part upon the mode of administration. Guidelines for choosing the combination of components for a quadruplex-targeted nucleic acid composition are known, and examples are set forth in U.S. Pat. Nos. 6,455,308 (Freier), 6,455,307 (McKay et al.), 6,451,602 (Popoffet al.), and 6,451,538 (Cowsert).

A quadruplex-targeted nucleic acid in the composition may be modified by chemical linkages, moieties, or conjugates that enhance activity, cellular distribution, or cellular uptake of the nucleic acid. Examples of such modifications are set forth in U.S. Pat. Nos. 6,455,308 (Freier), 6,455,307 (McKay et al.), 6,451,602 (Popoff et al.), and 6,451,538 (Cowsert).

A quadruplex-targeted nucleic acid compositions may be presented conveniently in unit dosage form, which is prepared according to conventional techniques known in the pharmaceutical industry. In general terms, such techniques include bringing a quadruplex-targeted nucleic acid into association with pharmaceutical carrier(s) and/or excipient(s) in liquid form or finely divided solid form, or both, and then shaping the product if required. The quadruplex-targeted nucleic acid compositions may be formulated into any dosage form, such as tablets, capsules, gel capsules, liquid syrups, soft gels, suppositories, and enemas. The compositions also may be formulated as suspensions in aqueous, non-aqueous, or mixed media. Aqueous suspensions may further contain substances which increase viscosity, including for example, sodium carboxymethylcellulose, sorbitol, and/or dextran. The suspension may also contain one or more stabilizers.

A quadruplex-targeted nucleic acid can be translocated into cells via conventional transformation or transfection techniques. As used herein, the terms “transformation” and “transfection” refer to a variety of standard techniques for introducing an aptamer into a host cell, which include calcium phosphate or calcium chloride co-precipitation, transduction/infection, DEAE-dextran-mediated transfection, lipofection, electroporation, and iontophoresis. Also, liposome compositions described herein can be utilized to facilitate quadruplex-targeted nucleic acid administration. A quadruplex-targeted nucleic acid composition may be administered to an organism in a number of manners, including topical administration (including ophthalmic and mucous membrane delivery (e.g., vaginal and rectal)), pulmonary administration (e.g., inhalation or insufflation of powders or aerosols, including by nebulizer; intratracheal, intranasal, epidermal and transdermal), oral administration, and parenteral administration (e.g., intravenous, intraarterial, subcutaneous, intraperitoneal injection or infusion, intramuscular injection or infusion; and intracranial (e.g., intrathecal or intraventricular)). In an embodiment, the composition is administered by colorectal delivery.

Utilization of MAX Quadruplex-Interacting Molecules

Because quadruplex forming nucleic acids are regulators of biological processes such as oncogene transcription, modulators of quadruplex biological activity can be utilized as cancer therapeutics. For example, molecules that interact with quadruplex structures can exert a therapeutic effect for certain cell proliferative disorders and related conditions because abnormally increased oncogene expression can cause cell proliferative disorders and quadruplex structures typically down-regulate oncogene expression. Quadruplex-interacting molecules can exert a biological effect according to different mechanisms, which include for example stabilizing a native quadruplex structure, inhibiting conversion of a native quadruplex to duplex DNA by blocking strand cleavage, and stabilizing a native quadruplex structure having an altered MAX quadruplex sequence. Also, administering a quadruplex forming nucleic acid having a similar or identical nucleotide sequence to a native oncogene regulating quadruplex sequence may act as a decoy by competing for cellular molecules that normally up-regulate an oncogene. Thus, quadruplex forming nucleic acids and quadruplex interacting molecules identified by the methods described herein may be administered to cells, tissues, or organisms for the purpose of down-regulating oncogene transcription and thereby treating cell proliferative disorders. The term “treatment” and “therapeutic effect” as used herein refer to reducing or stopping a cell proliferation rate (e.g. slowing or halting tumor growth) or reducing the number of proliferating cancer cells (e.g. removing part or all of a tumor).

Determining whether the biological activity of native quadruplex DNA is modulated in a cell, tissue, or organism can be accomplished by monitoring quadruplex biological activity. Quadruplex biological activity may be monitored in cells, tissues, or organisms, for example, by detecting a decrease or increase of gene transcription in response to contacting the quadruplex DNA with a molecule. Transcription can be detected by directly observing RNA transcripts or observing polypeptides translated by transcripts, which are methods well known in the art.

Quadruplex interacting molecules and quadruplex forming nucleic acids can be utilized to target many cell proliferative disorders. Cell proliferative disorders can include, but are not limited to cancers of the colorectum, breast, lung, liver, pancreas, lymph node, colon, prostate, brain, head and neck, skin, liver, kidney, and heart.

In a specific embodoment, MAX quadruplex interacting molecules may be used to enhance or modify the sensitivity of cancers to other anticancer agents. In specific embodiments, cancers identified as having an altering mutation in the MAX quadruplex sequence can be identified as those that are likely to benefit from combination therapeutic approaches. For example, in such cancers, compounds that modulate the MAX quadruplex can be combined with other agents that induce apoptosis such as avastin, dacarbazine (e.g., multiple myeloma), 5-FU (e.g., pancreatic cancer), gemcitabine (e.g., pancreatic cancer), gleevac (e.g., CML), and DNA-damaging agents. Also, compounds that modulate the MAX quadruplex can be combined with MAX interacting nucleic acids, such as an antisense nucleic acid, siRNA, RNAi or ribozyme for example.

Administering a molecule to an organism can be accomplished in a number of manners, including intradermal, intramuscular, intravenous, intraperitoneal, and subcutaneous administration. An effective amount of molecule for modulating the biological activity of native quadruplex DNA will depend in part on the molecule composition, the mode of administration, and the weight and general health of the organism, and can generally range from about 1.0 μg to about 5000 μg of peptide for a 70 kg patient. The effective amount can be optimized by determining whether the biological activity of the native quadruplex DNA is modulated in the system.

Thus, provided herein are methods for reducing cell proliferation or for treating or alleviating cell proliferative disorders by restoring or enhancing sensitivity to apoptotic stimuli, which comprise contacting a system having a native quadruplex DNA with a quadruplex interacting molecule or quadruplex forming nucleic acid identified by an assay described herein. The system sometimes is a group of cells or one or more tissues, and often is a subject in need of a treatment of a cell proliferative disorder (e.g., a mammal such as a mouse, rat, monkey, or human).

The invention is further illustrated by the following examples which should not be construed as limiting. The contents of the documents cited in this application are incorporated herein by reference.

### EXAMPLE 1

### Quadruplex Assays

Test molecules identified as quadruplex interacting molecules and quadruplex-forming nucleic acids often are further confirmed for quadruplex-forming activity or quadruplex-interacting activity in assays described hereafter. These assays include mobility shift assays, DMS methylation protection assays, polymerase arrest assays, transcription reporter assays, circular dichroism assays, and fluorescence assays.

Gel Electrophoretic Mobility Shift Assay (EMSA)

An EMSA is useful for determining whether a nucleic acid forms a quadruplex and whether a nucleotide sequence is quadruplex-altering. EMSA is conducted as described previously (Jin & Pike, Mol. Endocrinol. 10: 196-205 (1996)) with minor modifications. Synthetic single-stranded oligonucleotides are labeled in the 5′-terminus with T4-kinase in the presence of [α-³²P] ATP (1,000 mCi/mmol, Amersham Life Science) and purified through a sephadex column. ³²P-labeled oligonucleotides (˜30,000 cpm) then are incubated with or without various concentrations of a testing compound in 20 μl of a buffer containing 10 mM Tris pH 7.5, 100 mM KCl, 5 mM dithiothreitol, 0.1 mM EDTA, 5 mM MgCl₂, 10% glycerol, 0.05% Nonedit P-40, and 0.1 mg/ml of poly(dI-dC) (Pharmacia). After incubation for 20 minutes at room temperature, binding reactions are loaded on a 5% polyacrylamide gel in 0.25× Tris borate-EDTA buffer (0.25×TBE, 1×TBE is 89 mM Tris-borate, pH 8.0, 1 mM EDTA). The gel is dried and each band is quantified using a phosphorimager.

DMS Methylation Protection Assay

Chemical footprinting assays are useful for assessing quadruplex structure. Quadruplex structure is assessed by determining which nucleotides in a nucleic acid are protected or unprotected from chemical modification as a result of being inaccessible or accessible, respectively, to the modifying reagent. A DMS methylation assay is an example of a chemical footprinting assay. In such an assay, bands from EMSA are isolated and subjected to DMS-induced strand cleavage. Each band of interest is excised from an electrophoretic mobility shift gel and soaked in 100 mM KCl solution (300 μl) for 6 hours at 4° C. The solutions are filtered (microcentrifuge) and 30,000 cpm (per reaction) of DNA solution is diluted further with 100 mM KCl in 0.1×TE to a total volume of 70 μl (per reaction). Following the addition of 1 μl salmon sperm DNA (0.1 μg/μl), the reaction mixture is incubated with 1 μl DMS solution (DMS:ethanol; 4:1; v:v) for a period of time. Each reaction is quenched with 18 μl of stop buffer (b-mercaptoathanol:water:NaOAc (3 M); 1:6:7; v:v:v). Following ethanol precipitation (twice) and piperidine cleavage, the reactions are separated on a preparative gel (16%) and visualized on a phosphorimager.

Polymerase Arrest Assay

An example of the Taq polymerase stop assay is described in Han et al., Nucl. Acids Res. 27: 537-542 (1999), which is a modification of that used by Weitzmann et al., J. Biol. Chem. 271, 20958-20964 (1996). Briefly, a reaction mixture of template DNA (50 nM), Tris-HCl (50 mM), MgCl₂ (10 mM), DTT (0.5 mM), EDTA (0.1 mM), BSA (60 ng), and 5′-end-labeled quadruplex nucleic acid (˜18 nM) is heated to 90° C. for 5 minutes and allowed to cool to ambient temperature over 30 minutes. Taq Polymerase (1 μl) is added to the reaction mixture, and the reaction is maintained at a constant temperature for 30 minutes. Following the addition of 10 μl stop buffer (formamide (20 ml), 1 M NaOH (200 μl), 0.5 M EDTA (400 μl), and 10 mg bromophenol blue), the reactions are separated on a preparative gel (12%) and visualized on a phosphorimager. Adenine sequencing (indicated by “A” at the top of the gel) is performed using double-stranded DNA Cycle Sequencing System from Life Technologies. The general sequence for the template strands is TCCAACTATGTATAC-INSERT-TTAGCGACACGCAATTGCTATAGTGAGTCGTATTA. Bands on the gel that exhibit slower mobility are indicative of quadruplex formation.

Transcription Reporter Assay

A luciferase promoter assay described in He et al., Science 281: 1509-1512 (1998) often is utilized for the study of quadruplex formation. Specifically, a vector utilized for the assay is set forth in reference 11 of the He et al. document. In this assay, HeLa cells are transfected using the lipofectamin 2000-based system (Invitrogen) according to the manufacturer's protocol, using 0.1 μg of pRL-TK (Renilla luciferase reporter plasmid) and 0.9 μg of the quadruplex-forming plasmid. Firefly and Renilla luciferase activities are assayed using the Dual Luciferase Reporter Assay System (Promega) in a 96-well plate format according to the manufacturer's protocol.

Circular Dichroism Assay

Circular dichroism (CD) is utilized to determine whether another molecule interacts with a quadruplex nucleic acid. CD is particularly useful for determining whether a PNA or PNA-peptide conjugate hybridizes with a quadruplex nucleic acid in vitro. PNA probes are added to quadruplex DNA (5 μM each) in a buffer containing 10 mM potassium phosphate (pH 7.2) and 10 or 250 mM KCl at 37° C. and then allowed to stand for 5 min at the same temperature before recording spectra. CD spectra are recorded on a Jasco J-715 spectropolarimeter equipped with a thermoelectrically controlled single cell holder. CD intensity normally is detected between 220 nm and 320 nm and comparative spectra for quadruplex DNA alone, PNA alone, and quadruplex DNA with PNA are generated to determine the presence or absence of an interaction (see, e.g., Datta et al., JACS 123:9612-9619 (2001)). Spectra are arranged to represent the average of eight scans recorded at 100 nm/min.

Fluorescence Binding Assay

50 μl of quadruplex nucleic acid or a nucleic acid not capable of forming a quadruplex is added in 96-well plate. A test molecule or quadruplex-targeted nucleic acid also is added in varying concentrations. A typical assay is carried out in 100 μl of 20 mM HEPES buffer, pH 7.0, 140 mM NaCl, and 100 mM KCl. 50 μl of the signal molecule N-methylmesoporphyrin IX (NMM) then is added for a final concentration of 3 μM. NMM is obtained from Frontier Scientific Inc, Logan, Utah. Fluorescence is measured at an excitation wavelength of 420 nm and an emission wavelength of 660 nm using a FluroStar 2000 fluorometer (BMG Labtechnologies, Durham, N.C.). Fluorescence often is plotted as a function of concentration of the test molecule or quadruplex-targeted nucleic acid and maximum fluorescent signals for NMM are assessed in the absence of these molecules.

The contents of each document cited herein is incorporated by reference in its entirety.

### EXAMPLE 2

### Identification of Alleles Having an Altered MAX Quadruplex Sequence Associated with Cancer

Normal tissue and tumor specimens are collected from patients having tumors at the University of Arizona Cancer Center or other suitable source. Tissues arre embedded in paraffin and stored in a tissue bank. Paraffin blocks then are microtomed (i.e., thin slices were cut from each) and mounted on glass slides. Six slides are generated from each paraffin block, where two are used for orientation to determine where tumor and normal tissues are located. These slides are stained with hematoxylin and eosin. The other four are utilized for laser capture micro-dissection (LCM) and sequencing. LCM is utilized to collect cells from each tissue specimen. A PixCelII laser capture system (Arcturus) is utilized with the following settings: 30 μm, 50 mW power and 6.2 ms duration. Approximately 1500 pulses are taken and adhered to a CapSure HS LCM cap (Arcturus). A Pico Pure DNA extraction kit is used (Arcturus) to extract genomic DNA from the laser captured cells.

Extracted cells from primary tumor specimens are incubated in 10 μl of proteinase K solution for at least 16 hours at 65° C. The genomic DNA is used in subsequent PCR reactions with appropriate primers. Each 5 μl reaction contains 1× high-fidelity PCR buffer (Invitrogen), 50 μM each of dCTP, dATP, dGTP, and dTTP (Fermentas), 2 mM MgSO₄ (Invitrogen), 2.5 U platinum Taq high-fidelity polymerase (Invitrogen), 0.5 μM of each primer, distilled/deionized water, and 224 μl of the genomic DNA from above. The reactions are incubated in a DNA Engine Peltier Thermal Cycler as follows: 95° C., 5 minutes; (95° C., 1 minute; 59° C., 1 minute, 10 seconds; 72° C., 1 minute 30 seconds)×45; and then 72° C., 5 minutes. PCR products are held for a time at 4° C. and stored at −20° C. PCR products are resuspended in 100 μL of nuclease-free water and are sequenced using the appropriate primer and an ABI 377 automated sequencer (Applied Biosystems).

Allelic variants identified in the MAX-associated quadruplex forming DNA sequence are compared among normal or non-malignant samples and primary tumor samples. The presence of the allelic variants described above are useful for determining whether a subject is at risk of developing or having cancer.

### EXAMPLE 3

### Cancer Prognostic and Diagnostic Assay

A cancer prognostic or diagnostic assay is carried out by obtaining a DNA sample from a subject, determining the nucleotide sequence of a MAX-associated quadruplex-forming sequence, and identifying the subject as being at risk of developing or having cancer when the nucleotide sequence corresponds to an allele having an altered MAX quadruplex sequence. A tumor tissue sample is obtained for a subject and then DNA is extracted from the sample. The isolated DNA is quantified and then contacted with appropriate primers suitable for MAX quadruplex detection. Each 5 μl reaction contains 1× high-fidelity PCR buffer (Invitrogen), 50 μM each of dCTP, dATP, dGTP, and dTTP (Fermentas), 2 mM MgSO₄ (Invitrogen), 2.5 U platinum Taq high-fidelity polymerase (Invitrogen), 0.5 μM of each primer, distilled/deionized water, and 224 μl of the genomic DNA from above. The reactions are incubated in a DNA Engine Peltier Thermal Cycler as follows: 95° C., 5 minutes; (95° C., 1 minute; 59° C., 1 minute, 10 seconds; 72° C., 1 minute 30 seconds)×45; and then 72° C., 5 minutes. PCR products are held for a time at 4° C. and optionally stored at −20° C. PCR products are resuspended in 100 μL of nuclease-free water and sequenced using the appropriate primer and an ABI 377 automated sequencer. If one or more altering mutations in the MAX quadruplex sequence is identified, the subject is prognosed or diagnosed with cancer. The subject is identified as being at risk of developing or having cancer when an altered MAX quadruplex sequence is present in pre-malignant samples or in tumor samples.

### EXAMPLE 4

### Cancer Therapeutic

Performing the prognostic or diagnostic procedure described in Example 3, the presence of a cancer-associated allele can be detected. Where the allele has the destablizing mutation in the MAX quadruplex sequence, a PNA molecule having the neutralizing sequence is selected and utilized as a therapeutic. A PNA molecule 20 nucleotides in length or 15 amino acids in length and having a subsequence of the above nucleotide sequences also is utilized as a therapeutic.

PNAs are synthesized using an Applied Biosystems (Foster City, Calif.) Expedite 8909 Synthesizer using monomer Fmoc reagents from Applied Biosystems (Mayfield & Corey, Biorg. Med. Chem Lett. 9:1419-1422 (1999)). PNAs are purified by reverse-phase HPLC and analyzed by time-of-flight mass spectrometry (MALDI-TOF) as described in Mayfield & Corey, supra. PNA is quantified based on spectrophotometric A₂₆₀ values and the conversion factor of 30 μg/ml OD₂₆₀. The PNA molecule often is synthesized with a lysine moiety at the C-terminus.

PNA-peptide conjugates are optionally synthesized. PNA-peptide conjugates are synthesized with either the peptide or the PNA in the C-terminal position. Depending on the orientation, either the peptide is synthesized first by automated synthesis or the PNA is synthesized first by manual synthesis. After completion of this initial synthesis, a small aliquot is deprotected and cleaved, then characterized by MALDI-TOF spectrometry to ensure successful synthesis of the entire lot. Once the identity of the synthesis is confirmed, fully protected oligomer is used as the basis for addition of the PNA by manual synthesis or a peptide by automated synthesis. Boc-protected monomers normally are employed for PNA synthesis. When the PNA is added to the N-terminus of a peptide already prepared by Fmoc synthesis, Fmoc chemistry also may be used for the PNA synthesis. PNA-peptide conjugates are purified using the procedure described above or by using a Rainin HPLC system with a Dynamax detector set at 260 nm using a Delta Pak C18 300 Å column (7.8×300 mm) heated to 50° C. (see e.g. Wang et al., J. Mol. Biol. 313:933-940 (2001)). A peptide sometimes is conjugated to the PNA, and sometimes is an Antennapedia homeodomain peptide (i.e. GGRQIWFQNRMKWKK, GGLWFQNRMKWKKEN, GGGRQIKIWFQNRRMKWKK, or GGGKIWFQNRRMKWKKEN) or an HIV tat peptide (i.e. SEQ ID NO: 2-7 in U.S. Pat. No. 5,652,122). Where the N-terminus of the PNA is linked to the C-terminus of a peptide, the C-terminus of the PNA sometimes ends with a lysine moiety.

Fluorescent-labeled PNAs and PNA-peptide conjugates are optionally synthesized. Fluorescent-labeled PNAs and PNA-peptide conjugates are useful for detecting cells transfected with the PNA or PNA-peptide conjugate and for sorting transfected cells. PNA-peptide conjugates typically are labeled with fluorescein or rhodamine. Fluorescein maleimide is coupled to deprotected PNA-peptide conjugates through cysteine. Rhodamine can withstand trifluormethanesulfonic acid (TFMSA) cleavage conditions as well as four hours of TFA cleavage without breaking down and is added to the N-terminus of the fully protected PNA or PNA-peptide hybrid before cleavage. After the N-terminal Boc or Fmoc protecting group is removed from the completed PNA-peptide hybrid, rhodamine is coupled using diisopropylethtlamine (DIPEA) to increase the pH to 9.0. Coupling is complete after thirty minutes. At least a four-fold excess of rhodamine over the PNA-peptide is used, while fluorescein is used in twofold excess. After coupling, the finished product is washed extensively with DMF or NMP to remove the unreacted rhodamine.

The PNA or PNA-peptide conjugate then is transfected into cells in vitro or is delivered by intravenous administration to a subject. In either application, the PNA and PNA-peptide conjugates are formulated before delivery. In one application, PNA formulations are prepared by equilibrating 15 μl of 100 μM PNA in 135 μl of Opti-MEM (Life Technologies). In a separate tube, 4.5 μl of (7 μg/ml) LipofectAMINE (Life Technologies) is activated in 145.5 μl of Opti-MEM by vigorously shaking for 5 s followed by equilibration for 5-10 min at room temperature. LipofectAMINE is obtained from Life Technologies (Gaithersburg, Md.) and solubilized according to the manufacturer protocol in sterile water. The PNA and LipofectAMINE aliquots (300 μL each) are mixed together and agitated vigorously for 15 s. Lipid complexes are allowed to form by incubating the mixture at room temperature for 15-20 min in the dark. The solution containing the PNA-lipid complex (600 μl) is diluted to 3 ml with Opti-MEM to afford a solution containing 1 μM PNA. This solution then is diluted to a final working concentration, which is 100 nM in most cases.

For transfection in vitro, cells are plated at 11000-13000 cells/well in 48-well plates using Dulbecco's MEM (minimal essential media) with glutamine supplemented with 10% superstripped fetal calf serum, 20 mM HEPES buffer (final concentration, pH 7.4), 500 units/ml penicillin, 0.1 mg/ml streptomycin, and 0.06 mg/ml anti-PPLO reagent (Life Technologies). Superstripped serum is used to ensure that competing ligands are removed from serum prior to addition of molecules. Ligand stripping is achieved by twice extracting serum with activated charcoal and cation exchange (CAG 1-X8 resin, Bio-Rad, Hercules, Calif.). Superstripped serum is doubly filtered through a 0.2 μM filter prior to addition to media. Cells are incubated at 37° C. at 5% CO₂ for a minimum of 6 h prior to initiating transfection. The cells then are washed once with 250 μL of Opti-MEM, followed by overnight transfection with PNA-lipid complex.

In an alternative in vitro transfection procedure that does not utilize lipid-formulated PNA or PNA-peptide conjugates, cells are allowed to attach to 24-well plates in IX Dulbecco's Modified Eagle's Media (DMEM) (Mediatech, Hemdon, Va.) supplemented with 10% fetal bovine serum. Media is removed from cells and PNAs and conjugates are added directly for three minutes prior to addition of fresh media to bring the final concentration of oligomer to 1 μM. Cells are incubated for one to twelve hours, with maximal uptake observed after one hour. Following incubation, cells are rinsed 8-12 times with phosphate buffered saline (PBS) to remove residual free fluorescent material when fluorescent-tagged molecules are utilized. Cells then are treated with trypsin and transferred to Lab-TekII chamber slides (Nalge-Nunc, Rochester, N.Y.) for visualization. After reattachment, cells are washed several times with PBS and fixed with 70% methanol. Vectashield (Vector Laboratories, Burlingame, Calif.) mounting medium (25 μL) is added to the fixed slides. Cells are visualized using an Olympus BHS microscope with a reflected light fluorescence attachment.

Transfected cells optionally are analyzed by flow cytometry. Adherent populations of cells are treated with 1 μM rhodamine labeled PNA or PNA-conjugate for 2 h at 37° C. Cells are extensively washed, trypsinized, and resuspended in 0.5 ml 1×PBS. Populations are immediately analyzed on a FACStarPlus flow cytometer using LYSYS II software (Becton Dickinson, Franklin Lakes, N.J.) and a 575 nm broad band pass filter. Cell populations are gated to measure only fluorescence in intact cells.

1. An isolated nucleic acid 100 or fewer nucleotides in length comprising the nucleotide sequence
(SEQ ID NO:1)
5′-CGGCGGCGGGGAGGGGAAGGGGTGAAGGGGAGGGGGA-3′.

2. A method for identifying a compound that modulates the biological activity of a native MAX quadruplex DNA, which comprises
contacting a MAX quadruplex DNA with a candidate compound; and
determining the presence or absence of an interaction between the candidate compound and the quadruplex DNA, whereby the candidate compound that interacts with the quadruplex DNA is identified as the compound that modulates the biological activity of the native quadruplex DNA.

3. A method for identifying a compound that binds a native MAX quadruplex DNA, which comprises
contacting a MAX quadruplex DNA with a candidate compound; and
determining the presence or absence of binding between the candidate compound and the quadruplex DNA, whereby the candidate compound that binds the quadruplex DNA is identified as the compound that binds the native quadruplex DNA.

4. A method for modulating the biological activity of a native MAX quadruplex DNA, which comprises contacting a system comprising the native quadruplex DNA with a compound which interacts with a quadruplex DNA; whereby the compound modulates the biological activity of the native quadruplex DNA.

5. The method of any of claims 2-4, wherein the MAX quadruplex structure comprises
SEQ ID NO:1
5′-CGGCGGCGGGGAGGGGAAGGGGTGAAGGGGAGGGGGA-3′.


SEQUENCE LISTING (7 sequences)

SEQ ID NO: 1 - DNA, 37 bp, Artificial Sequence
  Primer
cggcggcggggaggggaaggggtgaaggggaggggga

SEQ ID NO: 2 - protein, 15 aa, Artificial Sequence
  Transduction peptide
GGRQIWFQNRMKWKK

SEQ ID NO: 3 - protein, 15 aa, Artificial Sequence
  Transduction peptide
GGLWFQNRMKWKKEN

SEQ ID NO: 4 - protein, 19 aa, Artificial Sequence
  Transduction peptide
GGGRQIKIWFQNRRMKWKK

SEQ ID NO: 5 - protein, 18 aa, Artificial Sequence
  Transduction peptide
GGGKIWFQNRRMKWKKEN

SEQ ID NO: 6 - DNA, 15 bp, Artificial Sequence
  Primer
tccaactatgtatac

SEQ ID NO: 7 - DNA, 35 bp, Artificial Sequence
  Primer
ttagcgacacgcaattgctatagtgagtcgtatta